(19) United States
(12) Patent Application Publication
(10) Pub. No.: US 2023/0111650 A1
(43) Pub. Date: Apr. 13, 2023

(54) Lymphocyte-Based PKCe Test for Alzheimer's Disease

(71) Applicant: NeuroDiagnostics LLC, Rockville, MD (US)

(72) Inventors: Florin Valentin Chirila, Morgantown, WV (US); Daniel L. Alkon, Chevy Chase, MD (US)

(73) Assignee: NeuroDiagnostics LLC, Rockville, MD (US)

(21) Appl. No.: 18/062,642

(22) Filed: Dec. 7, 2022

Related U.S. Application Data
(63) Continuation of application No. 15/939,841, filed on Mar. 29, 2018.
(60) Provisional application No. 62/584,381, filed on Nov. 10, 2017.
(60) Provisional application No. 62/479,663, filed on Mar. 31, 2017.

(51) Int. Cl. C12Q 1/48; G01N 33/50

(52) U.S. Cl. CPC C12Q 1/485; G01N 33/5091; G01N 33/5052; G01N 2333/912; G01N 2800/50; G01N 2800/2821

(57) ABSTRACT

This invention provides methods for diagnosing Alzheimer's disease in a symptomatic human subject, and for determining whether a human subject is predisposed to becoming afflicted with Alzheimer's disease. These methods involve the steps of (a) culturing lymphocytes from the subject under suitable conditions; (b) measuring the amount of PKCε in the cultured lymphocytes; and (c) comparing the measurement of step (b) with a suitable control.

This application claims the benefit of U.S. Provisional Applications No. 62/584,381, filed Nov. 10, 2017 and 62/479,663, filed Mar. 31, 2017, the contents of both of which are incorporated herein by reference.

Throughout this application, various publications are cited. The disclosure of these publications is hereby incorporated by reference into this application to describe more fully the state of the art to which this invention pertains.

### FIELD OF THE INVENTION

The present invention relates to PKCε-based methods for diagnosing Alzheimer's disease in a human subject, and for determining whether a human subject is predisposed to having Alzheimer's disease.

### BACKGROUND OF THE INVENTION

Many data have been collected over the last 15 years indicating that the pathophysiology of Alzheimer's disease is not just related to the brain, but can also have systemic expression. For example, many of the critical amyloid and tau enzymes can be found throughout the body. On this basis, accurate assays have been developed to test skin cells, for example, for Alzheimer's disease against cells from a variety of control patients. Some skin cell-based assays focus on targets such as PKCε and ERK_{1,2}.

### SUMMARY OF THE INVENTION

This invention provides a first method, namely, a method for diagnosing Alzheimer's disease in a symptomatic human subject comprising the steps of (a) culturing lymphocytes from the subject under conditions that preserve lymphocyte viability and/or permit the lymphocytes to proliferate; (b) measuring the amount of PKCε in the cultured lymphocytes; and (c) comparing the measurement of step (b) with a suitable control, thereby determining whether the subject is afflicted with Alzheimer's disease.

This invention also provides a second method, namely, a method for determining whether a human subject is predisposed to becoming afflicted with Alzheimer's disease comprising the steps of (a) culturing lymphocytes from the subject under conditions that preserve lymphocyte viability and/or permit the lymphocytes to proliferate; (b) measuring the amount of PKCε in the cultured lymphocytes; and (c) comparing the measurement of step (b) with a suitable control, thereby determining whether the subject is predisposed to becoming afflicted with Alzheimer's disease.

This invention further provides a third method, namely, a method for diagnosing Alzheimer's disease in a symptomatic human subject comprising the steps of (a) culturing two otherwise identical populations of lymphocytes from the subject under conditions that preserve lymphocyte viability and/or permit the lymphocytes to proliferate, the first population comprising a suitable concentration of amylospheroid and the second population being free of amylospheroid; (b) separately measuring the amount of PKCε in the cultured lymphocytes from the first and second populations; and (c) comparing the measurements of step (b), whereby the subject is afflicted with Alzheimer's disease if the amount of PKCε in the cultured lymphocytes from the first population is greater than or equal to the amount of PKCε in the cultured lymphocytes from the second population.

This invention also provides a fourth method, namely, a method for determining whether a human subject is predisposed to becoming afflicted with Alzheimer's disease comprising the steps of (a) culturing two otherwise identical populations of lymphocytes from the subject under conditions that preserve lymphocyte viability and/or permit the lymphocytes to proliferate, the first population comprising a suitable concentration of amylospheroid and the second population being free of amylospheroid; (b) separately measuring the amount of PKCε in the cultured lymphocytes from the first and second populations; and (c) comparing the measurements of step (b), whereby the subject is predisposed to becoming afflicted with Alzheimer's disease if the amount of PKCε in the cultured lymphocytes from the first population is greater than or equal to the amount of PKCε in the cultured lymphocytes from the second population.

This invention still further provides a fifth method, namely, a method for diagnosing Alzheimer's disease in a symptomatic human subject comprising the steps of (a) culturing two otherwise identical populations of lymphocytes from the subject under conditions that preserve lymphocyte viability and/or permit the lymphocytes to proliferate, the first population comprising a first suitable concentration of amylospheroid and the second population comprising a second suitable concentration of amylospheroid higher than the first concentration; (b) separately measuring the amount of PKCε in the cultured lymphocytes from the first and second populations; and (c) comparing the measurements of step (b), whereby the subject is afflicted with Alzheimer's disease if the amount of PKCε in the cultured lymphocytes from the second population is greater than the amount of PKCε in the cultured lymphocytes from the first population.

Finally, this invention provides a sixth method, namely, a method for determining whether a human subject is predisposed to becoming afflicted with Alzheimer's disease comprising the steps of (a) culturing two otherwise identical populations of lymphocytes from the subject under conditions that preserve lymphocyte viability and/or permit the lymphocytes to proliferate, the first population comprising a first suitable concentration of amylospheroid and the second population comprising a second suitable concentration of amylospheroid higher than the first concentration; (b) separately measuring the amount of PKCε in the cultured lymphocytes from the first and second populations; and (c) comparing the measurements of step (b), whereby the subject is predisposed to becoming afflicted with Alzheimer's disease if the amount of PKCε in the cultured lymphocytes from the second population is greater than the amount of PKCε in the cultured lymphocytes from the first population.

### BRIEF DESCRIPTION OF THE FIGURES

FIG. 1

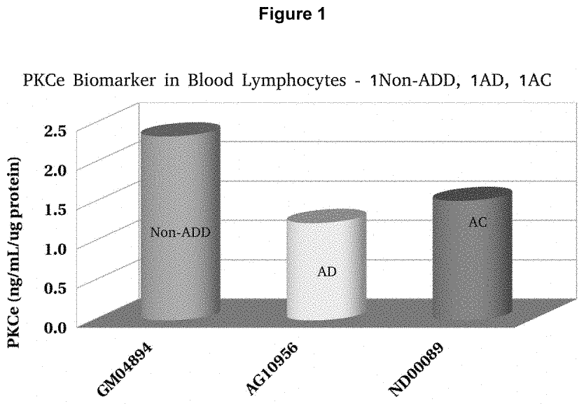

Low PKCe Level in Blood Lymphocytes

The PKCe level in an Alzheimer disease patient (AD—turquoise) is the lowest compared with the other two cases, a Huntington disease case (Non-ADD—light blue) and an Age-Matched Control (AC—purple). This result is analogous to the PKCe effect seen in skin fibroblasts and brain (Khan and Alkon, Journal of Alzheimer's Disease, 43 (2015) 491-509).

FIGS. 2A and 2B

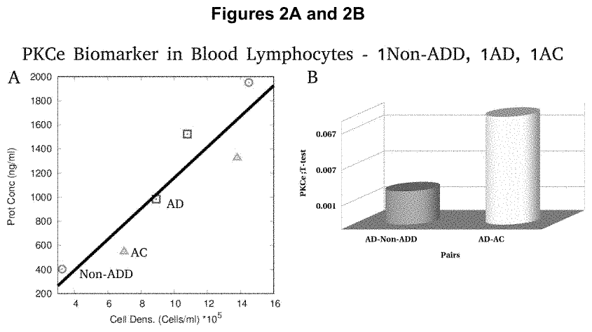

Linear Correlation of Total Protein Concentration with B Lymphocytes in the PKCe Biomarker

(FIG. 2A) The total protein concentration for B lymphocytes correlates linearly with the cell density (Pearson's linear correlation coefficient 0.916). (FIG. 2B) The PKCe level in the AD case is lower than the Non-ADD case with a statistical significance of P<0.001 (blue cylinder). The PKCe level in the AD case is not statistically significantly lower than the AC (yellow cylinder). However, the difference between AD and AC can be assessed clinically, as the AC cases are non-demented.

FIGS. 3A and 3B

Differential PKCe Values as a Function of ASPD Concentration

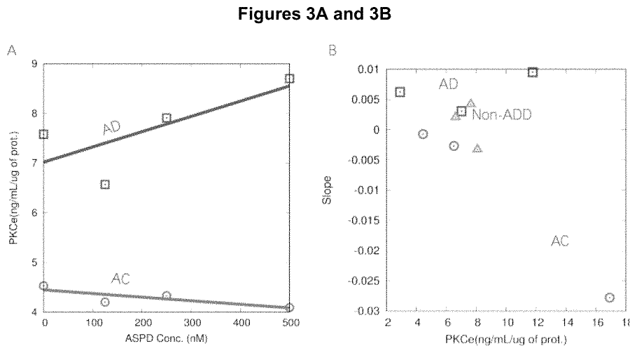

(FIG. 3A) PKCe values are shown for ASPD-treated B lymphocytes for an AD case (purple squares) and an AC case (green circles). Lines represent the best fit, (FIG. 3B) The distribution of the slopes (S) and intercepts (I) is shown for nine cell lines. Green circles represent AC. Purple squares represent AD. Blue triangles represent Non-ADD.

### DETAILED DESCRIPTION OF THE INVENTION

### Definitions

In this application, certain terms are used which shall have the meanings set forth as follows.

As used herein, “diagnosing Alzheimer's disease”, with respect to a symptomatic human subject, means determining that there is greater than 50% likelihood that the subject is afflicted with Alzheimer's disease. Preferably, “diagnosing Alzheimer's disease” means determining that there is greater than 60%, 70%, 80% or 90% likelihood that the subject is afflicted with Alzheimer's disease. As used herein, the phrase “determining whether the subject is afflicted with Alzheimer's disease” is synonymous with the phrase “diagnosing Alzheimer's disease.”

As used herein, “determining whether a human subject is predisposed to becoming afflicted with Alzheimer's disease” means determining that there is a greater than average likelihood that the subject will become afflicted with Alzheimer's disease during her or his lifetime. After the age of 65, one in five individuals will become afflicted with Alzheimer's disease, and after the age of 85, one in two individuals will become afflicted with Alzheimer's disease. Preferably, determining this predisposition means determining that there is at least a 20% likelihood that the subject will become afflicted with Alzheimer's disease during her or his lifetime.

As used herein, “Alzheimer's disease” means a concurrent affliction with the following three symptoms: (i) dementia; (ii) amyloid plaques; and (iii) neurofibrillary tangles. Dementia can be diagnosed during life. Cerebral amyloid plaques and neurofibrillary tangles can, for example, be diagnosed during autopsy. This definition of Alzheimer's disease is the one provided by the National Institute of Neurological Disorders and Stroke (NINDS) of the National Institutes of Health (NIH), and is known as the “gold standard.”

As used herein, a human subject who is “symptomatic” for Alzheimer's disease is a subject displaying at least one symptom of the disease, i.e., one of dementia, amyloid plaques, and neurofibrillary tangles. Preferably, a human subject who is symptomatic for Alzheimer's disease is a subject displaying dementia. Conversely, a human subject who is “asymptomatic” for Alzheimer's disease is a subject who does not display any symptom of the disease.

As used herein, the “human subject” can be of any age. In one embodiment, the subject is 40 years old or younger. In another embodiment, the subject is 50 years old or younger. In a further embodiment, the subject is over 40 years old. In yet a further embodiment, the subject is over 50 years old, over 60 years old, over 70 years old, over 80 years old, or over 90 years old.

As used herein, “culturing” lymphocytes under conditions permitting them to “proliferate” is achieved, for example, by conducting the culturing at a temperature and in a growth factor milieu permissive of cell growth. In another embodiment, “culturing” lymphocytes is performed under conditions (e.g., those described below for proliferation) that preserve lymphocyte viability. In one embodiment, the temperature, salinity and protein milieu permissive of cell growth is 37° C., RPMI 1640 Medium with 10% fetal bovine serum (“FBS”) and 1% penicillin (“PS”). In an unexpected feature of this invention, the lymphocytes (e.g., B lymphocytes) remain viable for an extended duration, thereby permitting lymphocyte PKCε measurement more than three hours after harvesting the lymphocytes. In one embodiment of this invention, the lymphocyte-culturing step is performed for more than three hours. Preferably, the lymphocyte-culturing step is performed for more than six hours (e.g., overnight). In immortalized B lymphocytes, the PKCε levels remain stable indefinitely and thus can be measured days, weeks, months or years after immortalization.

As used herein, culturing lymphocytes “from” a subject means culturing lymphocytes originating from the subject, wherein prior to culturing, the lymphocytes either have or have not been manipulated (e.g., isolated, immortalized and/or otherwise modified) following removal from the subject.

As used herein, “lymphocytes” include, by way of example, B lymphocytes, T lymphocytes, and mixtures thereof. Preferably, the lymphocytes are B lymphocytes. Methods for obtaining lymphocytes from a subject's blood are known, and include, for example, flow cytometry, Ficoll (a hydrophilic polysaccharide that separates layers of blood), and gradient centrifugation. Additionally, in the subject methods, the lymphocytes (e.g., B lymphocytes) can be used in immortalized or primary (i.e., non-immortalized) form. Methods for immortalizing lymphocytes (e.g., B lymphocytes) are known, and include, for example, treating the lymphocytes with Epstein-Barr virus (“EBV”).

As used herein, “PKCε” (also referred to herein as “PKCe”) shall mean protein kinase C epsilon.

As used herein, “measuring” the amount of PKCε in the cultured lymphocytes can be performed, for example, in situ or after isolating protein from the lymphocytes. Moreover, the measuring can be performed, for example, using any method by which the amount of a protein kinase can be quantitatively determined. Such methods are known and include, without limitation, ELISA-based assays, spectrophotometric assays, Western blot methods, Duolink methods, and any other antibody-based method. In one embodiment, measuring the amount of PKCε in cultured lymphocytes means determining the amount of PKCε as expressed, for example, in (i) fg of PKCε per lymphocyte, (ii) ng of PKCε per known population of cells, (iii) ng of PKCε per volume of cell digest, or (iv) ng of PKCε per volume of supernatant generated during the PKCε isolation process. In another embodiment, such measurements are expressed in relation to total lymphocyte protein. For example, the amount of PKCε measured in cultured lymphocytes can be expressed as “ng/mL/μg”, wherein “mL” represents the volume of supernatant generated during the PKCε isolation process, “ng” represents the amount of PKCε present in the supernatant, and “μg” represents the amount of protein present in the supernatant. In a further embodiment, measuring the amount of PKCε in cultured lymphocytes means determining the rate of change over time in the amount of PKCε as expressed, for example, in one or more of the units described in this paragraph.

As used herein, a “suitable control” for performing the subject methods requiring same includes, without limitation, a positive control, a negative control, or one or more of each. For example, a positive control could be lymphocyte PKCε data obtained by performing one of the subject methods on lymphocytes obtained from a human subject afflicted with Alzheimer's disease. A negative control could be, for example, lymphocyte PKCε data obtained by performing one of the subject methods on lymphocytes obtained from a human subject who is not afflicted with Alzheimer's disease (e.g., a subject without any cognitive symptoms, a subject afflicted with mild cognitive impairment, or a subject afflicted with non-Alzheimer's dementia). Importantly, it is envisioned that in some of the subject methods, control tests (e.g., positive, negative and/or both) will be performed before, concurrently with or after testing the lymphocytes from the human subjects of interest. The data from such control tests can then serve as suitable controls. It is also envisioned that in other of the subject methods, no control tests are performed before, concurrently with or after testing the lymphocytes from the human subjects of interest. Instead, in each such method, determining affliction with, or predisposition to, Alzheimer's disease (as applicable) can be achieved, for example, by comparing the method's result with lymphocyte PKCε parameters (e.g., 1.0 ng/mL/μg) having a known correlation with disease state or predisposition, as applicable.

As used herein, an “amylospheroid” (also referred to herein as “ASPD”) means a spherical aggregate of β-amyloid. Amylospheroids are known and commercially available. Preferably, the amylospheroid is of a toxic size, such as having a diameter of greater than 10 nm (e.g., 10 nm to 20 nm, 20 nm to 50 nm, and 50 nm to 100 nm).

As used herein, a “suitable concentration” of amylospheroid includes, without limitation, greater than 0 nM and less than 500 nM (e.g., 1 nM to 100 nM, 100 nM to 200 nM, 200 nM to 300 nM, 300 nM to 400 nM, and 400 nM to 500 nM).

As used herein, “separately measuring”, with respect to the amount of PKCε in two different cultured lymphocyte populations, means measuring the amount of PKCε in each population without first mixing or otherwise combining the two populations.

### Embodiments of the Invention

This invention provides lymphocyte-based methods for diagnosing Alzheimer's disease in a human subject. It also provides lymphocyte-based methods for determining whether a human subject is predisposed to becoming afflicted with Alzheimer's disease. The subject methods are based, at least in part, on the surprising discovery that a subject's lymphocytes can be used in a PKCε-based test to either diagnose Alzheimer's disease or determine a predisposition to Alzheimer's disease, as applicable.

Specifically, this invention provides a first method, namely, a method for diagnosing Alzheimer's disease in a symptomatic human subject comprising the steps of (a) culturing lymphocytes from the subject under conditions that preserve lymphocyte viability and/or permit the lymphocytes to proliferate; (b) measuring the amount of PKCε in the cultured lymphocytes; and (c) comparing the measurement of step (b) with a suitable control, thereby determining whether the subject is afflicted with Alzheimer's disease.

In this first method and the second, third, fourth, fifth and sixth methods described below, the lymphocytes can be any type of lymphocytes. Preferably, the lymphocytes are B lymphocytes. Also preferred are B lymphocytes that are immortalized.

In this first method and the second, third, fourth, fifth and sixth methods described below, wherein culturing step (a) is performed for more than three hours, and ideally for more than six hours.

In a preferred embodiment of this first method and the third and fifth methods described below, the symptomatic human subject is suspected of having either Alzheimer's disease or non-Alzheimer's dementia, and the method permits determining with which of these two disorders the subject is afflicted.

In a preferred embodiment of this first method and the second, third, fourth, fifth and sixth methods described below, this invention provides a method wherein step (a) comprises culturing immortalized B lymphocytes from the subject for more than six hours.

This invention also provides a second method, namely, a method for determining whether a human subject is predisposed to becoming afflicted with Alzheimer's disease comprising the steps of (a) culturing lymphocytes from the subject under conditions that preserve lymphocyte viability and/or permit the lymphocytes to proliferate; (b) measuring the amount of PKCε in the cultured lymphocytes; and (c) comparing the measurement of step (b) with a suitable control, thereby determining whether the subject is predisposed to becoming afflicted with Alzheimer's disease.

In this second method and the fourth and sixth methods described below, the subject may be afflicted with a cognitive disability. In one embodiment, this disability is mild cognitive impairment. Alternatively, in this second method and the fourth and sixth methods described below, the subject is not cognitively impaired.

This invention further provides a third method, namely, a method for diagnosing Alzheimer's disease in a symptomatic human subject comprising the steps of (a) culturing two otherwise identical populations of lymphocytes from the subject under conditions that preserve lymphocyte viability and/or permit the lymphocytes to proliferate, the first population comprising a suitable concentration of amylospheroid and the second population being free of amylospheroid; (b) separately measuring the amount of PKCε in the cultured lymphocytes from the first and second populations; and (c) comparing the measurements of step (b), whereby the subject is afflicted with Alzheimer's disease if the amount of PKCε in the cultured lymphocytes from the first population is greater than or equal to the amount of PKCε in the cultured lymphocytes from the second population.

This invention also provides a fourth method, namely, a method for determining whether a human subject is predisposed to becoming afflicted with Alzheimer's disease comprising the steps of (a) culturing two otherwise identical populations of lymphocytes from the subject under conditions that preserve lymphocyte viability and/or permit the lymphocytes to proliferate, the first population comprising a suitable concentration of amylospheroid and the second population being free of amylospheroid; (b) separately measuring the amount of PKCε in the cultured lymphocytes from the first and second populations; and (c) comparing the measurements of step (b), whereby the subject is predisposed to becoming afflicted with Alzheimer's disease if the amount of PKCε in the cultured lymphocytes from the first population is greater than or equal to the amount of PKCε in the cultured lymphocytes from the second population.

This invention further provides a fifth method, namely, a method for diagnosing Alzheimer's disease in a symptomatic human subject comprising the steps of (a) culturing two otherwise identical populations of lymphocytes from the subject under conditions that preserve lymphocyte viability and/or permit the lymphocytes to proliferate, the first population comprising a first suitable concentration of amylospheroid and the second population comprising a second suitable concentration of amylospheroid higher than the first concentration; (b) separately measuring the amount of PKCε in the cultured lymphocytes from the first and second populations; and (c) comparing the measurements of step (b), whereby the subject is afflicted with Alzheimer's disease if the amount of PKCε in the cultured lymphocytes from the second population is greater than the amount of PKCε in the cultured lymphocytes from the first population.

This invention still further provides a sixth method, namely, a method for determining whether a human subject is predisposed to becoming afflicted with Alzheimer's disease comprising the steps of (a) culturing two otherwise identical populations of lymphocytes from the subject under conditions that preserve lymphocyte viability and/or permit the lymphocytes to proliferate, the first population comprising a first suitable concentration of amylospheroid and the second population comprising a second suitable concentration of amylospheroid higher than the first concentration; (b) separately measuring the amount of PKCε in the cultured lymphocytes from the first and second populations; and (c) comparing the measurements of step (b), whereby the subject is predisposed to becoming afflicted with Alzheimer's disease if the amount of PKCε in the cultured lymphocytes from the second population is greater than the amount of PKCε in the cultured lymphocytes from the first population.

In the fifth and sixth methods, the first and second suitable concentrations of amylospheroid (again, represented by “ASPD”) include, for example, values ranging from 0 nM to 500 nM, with the proviso that at least the second amylospheroid concentration is greater than 0 nM. Preferably, the first and second suitable concentrations of ASPD include values ranging from 0 nM to 125 nM, as exemplified by the following two-concentration permutations: (i) 0 nM and 25 nM; (ii) 0 nM and 50 nM; (iii) 0 nM and 75 nM; (iv) 0 nM and 100 nM; and (v) 0 nM and 125 nM.

In one embodiment of the fifth and sixth methods, the method comprises culturing more than two otherwise identical populations of lymphocytes from the subject, each population comprising a unique suitable concentration of ASPD, and separately measuring the amount of PKCε in the cultured lymphocytes from each population. Examples of first, second and third suitable concentrations of ASPD, to name just one embodiment, include the following: (i) 0 nM, 25 nM and 50 nM; (ii) 0 nM, 50 nM and 100 nM; and (iii) 0 nM, 50 nM and 125 nM.

The diagnostic outcome of the fifth method, and the prognostic outcome of the sixth method, can be expressed, in one embodiment, by plotting the PKCε measurements (i.e., data points) on a graph and measuring the slope of the line formed by the data points. As a specific example, when the PKCε measurements are plotted on a graph wherein the x-axis=ASPD concentration (nM) (e.g., 0 nM to 100 nM, 125 nM, 200 nM, 300 nM, 400 nM or 500 nM) and the y-axis=PKCε (ng/mL/μg of protein), the resulting slope is positive (e.g., greater than 0.000, greater than 0.005, or greater than 0.010). Preferably, each data point plotted is an averaged value.

Finally, this invention provides a kit for performing any of the subject methods, wherein the kit comprises, in separate compartments or a single compartment, (i) a growth medium (e.g., RPMI 1640 Medium with 10% FBS and 1% PS) and (ii) a means for quantitatively measuring PKCε (e.g., a PKCε ELBA kit with sandwich human antibody). The kit may optionally contain ASPD suitably formulated for performing the subject ASPD-dependent methods. PKCε ELISA kits are commercially available (e.g., https://www.mybiosource.com/prods/ELISA-Kit/Human/Protein-Kinase-C-Epsilon/PKCE/datasheet.php?product_id=21356 or http://www.antibodies-online.com/search.php).

Also envisioned is the application of the instant invention to (i) any non-lymphocyte peripheral blood mononuclear cell (PBMC) and (ii) any pluripotent stem cell (iPSC) derived from a reprogrammed EBV-immortalized B lymphocyte (https://www.ncbi.nlm.nih.gov/pmc/articles/PMC3158714/; and https://www.ncbi.nlm.nih.gov/pmc/articles/PMC2596471/). In this embodiment, such cells are treated, mutatis mutandis, as lymphocytes are treated in this invention.

This invention will be better understood by reference to the examples which follow, but those skilled in the art will readily appreciate that the specific examples detailed are only illustrative of the invention as described more fully in the claims which follow thereafter.

### EXAMPLE 1

PBMC Preparation. Whole blood, sampled in BD Vacutainer CPTTM Cell Preparation Tubes with sodium heparin (BD Bioscience, Franklin Lakes, N.J., USA), was diluted 1:2 in PBS and gently layered over cold Ficoll-Paque PLUS (GE Healthcare, Little Chalfont, UK) and kept on ice before centrifugation for 20 min at 900×g without brake at 18-20° C. The cell layer on top of the Ficoll-Paque PLUS consisting of peripheral blood mononuclear cells (PBMCs) was collected and diluted in PBS. PBMCs were centrifuged again for 7 min at 450×g at 18-20° C. The supernatant was removed and cells were re-suspended in 5 ml PBS and counted before they were centrifuged at 350×g for seven min at 18-20° C. Finally, cells were re-suspended in freezing medium (10% DMSO in heat-inactivated fetal bovine serum (FBS) (Nordic Biolabs Aft Taby, Sweden) and placed at −70° C. in a Mr. Frosty™ Freezing Container (Thermo Fisher) for at least three hours before being transferred to liquid nitrogen for extended storage.

Flow Cytometry. For each individual cell sample, 5×10⁶ PBMCs were quickly thawed at 37° C. and diluted in 40 ml cold wash buffer (PBS supplemented with 2.5% FBS (Life Technologies Ltd. Paisley, UK) and 0.1% sodium azide). The cell suspension was centrifuged at 250×g for five min and after discarding the supernatant, the pellet was re-suspended in 400 ml wash buffer. Each sample was stained for one hour in a light-protected environment at 2° C. with titrated amounts of anti-CD19 labeled with Alexa Fluor 700 (BD Biosciences). Samples were analyzed using a BD LSR II Special Order System, controlled by the BD FACSDiva 6.0 software (BD Biosciences). A preliminary forward scatter (FSC) versus side scatter (SSC) gate was used to identify lymphocytes and, depending on sample size, a total of up to 100,000 in-gate events were recorded. All datasets were migrated to FlowJo 7.6.5 (Treestar Inc. Ashland, Oreg., USA) for further gating and analysis.

[symbol]

Cells were cultured as per Coriell Cell Repository specifications, at 37° C., in water jacket CO₂ incubators in RPMI 1640 Medium, with 10% FBS and 1% PS.

1.-42. (canceled)

43. A method comprising the step of culturing two otherwise identical populations of human B lymphocytes for more than three hours under conditions that preserve B lymphocyte viability and/or permit the B lymphocytes to proliferate, the first population comprising a suitable concentration of amylospheroid and the second population being free of amylospheroid, wherein the B lymphocytes are from a human subject symptomatic of either Alzheimer's disease or non-Alzheimer's dementia.

44. The method of claim 43, wherein the B lymphocytes are immortalized.

45. The method of claim 43, wherein the culturing is performed for more than six hours.

46. A method comprising the step of culturing two otherwise identical populations of human B lymphocytes for more than three hours under conditions that preserve B lymphocyte viability and/or permit the B lymphocytes to proliferate, the first population comprising a first suitable concentration of amylospheroid and the second population comprising a second suitable concentration of amylospheroid higher than the first concentration, wherein the B lymphocytes are from a human subject symptomatic of either Alzheimer's disease or non-Alzheimer's dementia.

47. The method of claim 46, wherein the B lymphocytes are immortalized.

48. The method of claim 46, wherein the culturing is performed for more than six hours.
